(19) United States
(12) Patent Application Publication
(10) Pub. No.: US 2026/0015382 A1
(43) Pub. Date: Jan. 15, 2026

(54) METHODS FOR TREATING POLYSORBATE-CONTAINING PROTEIN FORMULATIONS

(71) Applicant: Janssen Biotech, Inc., Horsham, PA (US)

(72) Inventors: Maurice De Jonker, Wassenaar (NL); Vincentius Andrianto Halim, Utrecht (NL); Clara Esteve Gil, Leiden (NL); Crina Balog, Woubrugge (NL)

(21) Appl. No.: 18/996,038

(22) PCT Filed: Jul. 27, 2023
(86) PCT No.: PCT/IB2023/057635

§ 371 Date: Jan. 17, 2025

Related U.S. Application Data
(60) Provisional application No. 63/393,575, filed on Jul. 29, 2022.

(51) Int. Cl. C07K 1/30; G01N 30/02; G01N 30/74; G01N 30/88

(52) U.S. Cl. CPC C07K 1/30; G01N 30/74; G01N 30/88; G01N 2030/027; G01N 2030/884

(57) ABSTRACT

Provided herein are methods of precipitating proteins within compositions that contain a protein and a polysorbate. These methods allow for an accurate determination of the concentration or the amount of polysorbate within the composition.

### CROSS REFERENCE TO RELATED APPLICATIONS

This application claims the benefit of U.S. Provisional Application 63/393,575, filed on Jul. 29, 2022, which is incorporated by reference herein in it's entirety.

### TECHNICAL FIELD

Disclosed herein are methods of precipitating proteins from polysorbate-containing protein formulations.

### BACKGROUND

Non-ionic surfactants such as Polysorbate 20/80 (PS20/PS80), commercially known as Tween 20/80, are commonly used in protein drug formulations to increase protein stability from surface stress and shear stress. Polysorbate degradation has gained attention across the industry because it may result in less stable products and the formation of sub-visible and/or visible particles in the drug product during its shelf-life, raising a concern for patient safety. Therefore, it is important to monitor polysorbate concentration in drug product formulations. But for samples containing high amounts of protein, existing methods for analyzing polysorbate concentrations can be negatively affected by several factors including high sample viscosity, gel formation, column clogging, interfering peaks and inaccurate result.

### SUMMARY

Disclosed herein are methods for precipitating a protein from a polysorbate-containing composition, comprising adding to the polysorbate-containing composition that comprises a protein and a polysorbate, an amount of a chelating agent and an amount of a C1-C6 alcohol that together are effective to precipitate the protein.

### BRIEF DESCRIPTION OF THE DRAWINGS

The summary, as well as the following detailed description, is further understood when read in conjunction with the appended drawings. For the purpose of illustrating the disclosed methods, the drawings show exemplary embodiments of the methods; however, the methods are not limited to the specific embodiments disclosed. In the drawings:

FIG. 1 shows a schematic diagram of an exemplary EDTA/EtOH precipitation scheme (black square) prior to polysorbate analysis with mixed-mode anion exchange-hydrophobic (MAX) high-performance liquid chromatography (HPLC) using an evaporative light scattering detector (ELSD). The volumes can be proportionally scaled.

FIG. 2A shows a MAX-ELSD chromatogram of a PS80 standard (400 g/mL).

FIG. 2B shows a MAX-ELSD chromatogram of an Antibody 4 drug product (containing 400 g/mL PS80). FIG. 2C shows a MAX-ELSD chromatogram of an EDTA/EtOH precipitated Antibody 4 drug product (containing 400 g/mL PS80).

### DETAILED DESCRIPTION OF ILLUSTRATIVE EMBODIMENTS

The disclosed methods may be understood more readily by reference to the following detailed description taken in connection with the accompanying figures, which form a part of this disclosure. It is to be understood that the disclosed methods are not limited to the specific methods described and/or shown herein, and that the terminology used herein is for the purpose of describing particular embodiments by way of example only and is not intended to be limiting of the claimed methods.

Unless specifically stated otherwise, any description as to a possible mechanism or mode of action or reason for improvement is meant to be illustrative only, and the disclosed methods are not to be constrained by the correctness or incorrectness of any such suggested mechanism or mode of action or reason for improvement.

Where a range of numerical values is recited or established herein, the range includes the endpoints thereof and all the individual integers and fractions within the range, and also includes each of the narrower ranges therein formed by all the various possible combinations of those endpoints and internal integers and fractions to form subgroups of the larger group of values within the stated range to the same extent as if each of those narrower ranges was explicitly recited. Where a range of numerical values is stated herein as being greater than a stated value, the range is nevertheless finite and is bounded on its upper end by a value that is operable within the context of the herein disclosure. Where a range of numerical values is stated herein as being less than a stated value, the range is nevertheless bounded on its lower end by a non-zero value. It is not intended that the scope of the methods be limited to the specific values recited when defining a range. All ranges are inclusive and combinable.

When values are expressed as approximations, by use of the antecedent “about,” it will be understood that the particular value forms another embodiment. Reference to a particular numerical value includes at least that particular value, unless the context clearly dictates otherwise.

It is to be appreciated that certain features of the disclosed methods which are, for clarity, described herein in the context of separate embodiments, may also be provided in combination in a single embodiment. Conversely, various features of the disclosed methods that are, for brevity, described in the context of a single embodiment, may also be provided separately or in any subcombination.

As used herein, the singular forms “a,” “an,” and “the” include the plural.

Various terms relating to aspects of the description are used throughout the specification and claims. Such terms are to be given their ordinary meaning in the art unless otherwise indicated. Other specifically defined terms are to be construed in a manner consistent with the definitions provided herein.

The term “comprising” is intended to include examples encompassed by the terms “consisting essentially of” and “consisting of”; similarly, the term “consisting essentially of” is intended to include examples encompassed by the term “consisting of.”

Disclosed herein are methods for precipitating a protein from a polysorbate-containing composition, comprising adding to the polysorbate-containing composition that comprises a protein and a polysorbate, an amount of a chelating agent and an amount of a C1-C6 alcohol that together are effective to precipitate said protein. The method may comprise providing a composition that comprises a protein and a polysorbate and adding to the composition an amount of a chelating agent and an amount of C1-C6 alcohol that is effective to precipitate the protein.

The chelating agent can be added to the composition before the alcohol is added to the composition. The chelating agent and the alcohol can be added to the composition at the same time.

Suitable chelating agents include one or more of ethylenediaminetetraacetic acid (EDTA), triethylamine (TEA), (ethylene glycol-bis(β-aminoethyl ether)-N,N,N′,N′-tetraacetic acid) (EGTA), n-(2-hydroxyethyl)ethylenediamine-N,N′,N (HEDTA), nitrilotriacetic acid (NTA), and 2-hydroxybenzoic acid (salicylic acid; SA). In some embodiments, the chelating agent is ethylenediaminetetraacetic acid (EDTA). In some embodiments, the chelating agent is triethylamine (TEA). In some embodiments, the chelating agent is (ethylene glycol-bis(β-aminoethyl ether)-N,N,N′,N′-tetraacetic acid) (EGTA). In some embodiments, the chelating agent is n-(2-hydroxyethyl)ethylenediamine-N,N′,N (HEDTA). In some embodiments, the chelating agent is nitrilotriacetic acid (NTA). In some embodiments, the chelating agent is 2-hydroxybenzoic acid (salicylic acid; SA). In some embodiments, the chelating agent is any combination of ethylenediaminetetraacetic acid (EDTA), triethylamine (TEA), (ethylene glycol-bis(β-aminoethyl ether)-N,N,N′,N′-tetraacetic acid) (EGTA), n-(2-hydroxyethyl)ethylenediamine-N,N′,N (HEDTA), nitrilotriacetic acid (NTA), and 2-hydroxybenzoic acid (salicylic acid; SA).

The concentration of chelating agent can be about 5 mM to about 50 mM. Suitable concentrations of the chelating agent can be, for example, about 5 mM, about 10 mM, about 15 mM, about 20 mM, about 25 mM, about 30 mM, about 35 mM, about 40 mM, about 45 mM, or about 50 mM. In some embodiments, the concentration of chelating agent is 15 mM.

Suitable C1-C6 alcohols include methanol, ethanol, propanol, butanol, pentanol, and hexanol. In some embodiments, the C1-C6 alcohol is a methanol. In some embodiments, the C1-C6 alcohol is an ethanol. In some embodiments, the C1-C6 alcohol is a propanol. The propanol can be iso-propanol. In some embodiments, the C1-C6 alcohol is a butanol. In some embodiments, the C1-C6 alcohol is a pentanol. In some embodiments, the C1-C6 alcohol is a hexanol. In some embodiments, the C1-C6 alcohol is any combination of methanol, ethanol, propanol, butanol, pentanol, and hexanol.

The concentration of alcohol can be about 45% to about 55%. Suitable concentrations of the alcohol can be, for example, about 45%, about 46%, about 47%, about 48%, about 49%, about 50%, about 51%, about 52%, about 53%, about 54%, or about 55%. In some embodiments, the alcohol concentration is about 50% by volume. The concentration of alcohol added to the composition can be any concentration effective to precipitate the proteins within the composition.

The disclosed methods can comp rise a C1-C6 alcohol that is ethanol together with a chelating agent selected from EDTA, TEA, EGTA, HEDTA, NTA, and salicylic acid. The disclosed methods can comprise adding to the composition comprising a protein and a polysorbate an amount of both: ethanol and EDTA; ethanol and TEA; ethanol and EGTA; ethanol and HEDTA; ethanol and NTA; or ethanol and salicylic acid, that together are effective to precipitate the protein. In some embodiments, the C1-C6 alcohol is ethanol and the chelating agent is EDTA. The methods can comprise adding to the composition that comprises a protein and a polysorbate, an amount of EDTA and an amount of ethanol that together are effective to precipitate the protein.

The protein can be an antibody. In some embodiments the antibody is a monoclonal antibody.

The methods can further comprise removing the protein from the composition. Removing the protein from the composition can comprise precipitating the protein, centrifuging the composition to pellet the precipitated protein, and separating the supernatant from the precipitated protein to thereby remove the protein from the composition. The methods can further comprise determining the concentration or amount of the polysorbate in the composition. Prior to determining a concentration or an amount of the polysorbate, the precipitated protein can be separated from the non-precipitated portion thereby forming a supernatant. In some embodiments, the chelating agent, alcohol, and the composition comprising protein and polysorbate are mixed prior to separating the precipitated protein from the non-precipitated portion. In some embodiments, the precipitated protein is separated from the non-precipitated portion via centrifugation. The centrifugation can be performed until the supernatant is essentially free of the precipitated protein. Supernatants essentially free of the precipitated protein can comprise a residual protein concentration that is about 100 times less than the initial protein concentration prior to precipitation and centrifugation.

The disclosed methods can be performed on compositions comprising greater than about 10 mg/mL of protein. Protein concentration can be about 10 mg/mL to about 250 mg/mL. Suitable protein concentrations can be, for example, about 10 mg/mL, about 20 mg/mL, about 30 mg/mL, about 40 mg/mL, about 50 mg/mL, about 60 mg/mL, about 70 mg/mL, about 80 mg/mL, about 90 mg/mL, about 100 mg/mL, about 110 mg/mL, about 120 mg/mL, about 130 mg/mL, about 140 mg/mL, about 150 mg/mL, about 160 mg/mL, about 170 mg/mL, about 180 mg/mL, about 190 mg/mL, about 200 mg/mL, about 210 mg/mL, about 220 mg/mL, about 230 mg/mL, about 240 mg/mL, about 250 mg/mL. In some embodiments, the protein concentration is about 20 mg/mL of protein.

The methods can further comprise determining a concentration of polysorbate in a non-precipitated portion of the composition. In some embodiments, the methods further comprise determining an amount of said polysorbate in a non-precipitated portion of the composition. For example, the methods can comprise precipitating the protein, centrifuging the composition to pellet the precipitated protein, and determining the concentration/amount of polysorbate in the supernatant. Polysorbate can be measured by any method know in the art. In some embodiments, the concentration or amount of polysorbate is determined via mixed-mode anion exchange-hydrophobic high-performance liquid chromatography using an evaporative light scattering detector (MAX-ELSD). During MAX-ELSD, an acidified mobile phase causes positively charged proteins to elute in the void volume, while neutral polysorbate molecules are retained and eluted separately from the protein. In some embodiments, the concentration or amount of polysorbate is determined via mixed-mode anion exchange-hydrophobic high-performance liquid chromatography using a charged aerosol detector (MAX-CAD).

### EXAMPLES

Polysorbate is a common stabilizer used in protein drug formulations. However, degradation of polysorbate may result in less stable product and the formation of sub-visible and/or visible particles, which might not be suitable for administration to patients. To evaluate if the amount of polysorbate in antibody-containing formulations can be accurately measured using chromatography, various antibody-containing formulations were subjected to initial chromatographic conditions by exposing those formulations to an aqueous solution containing 10% isopropanol and 2% formic acid. Table 1 lists 11 antibodies, their respective protein concentrations, and whether they formed a gel upon mixing with acidified isopropanol, which mimics the chromatographic conditions experienced by the samples during analysis. Seven of the 11 antibody-containing formulations, each of which contained protein concentrations above 100 mg/mL, formed a gel under these conditions. These results demonstrate the risk of protein gel formation during high-performance liquid chromatography (HPLC).

TABLE 1
Evaluation of gel formation in antibody-containing formulations
following exposure to the initial chromatographic conditions
(in 10% isopropanol/2% formic acid).

| | Protein Concentration | Gel |
|---|---|---|
| Antibodies | (mg/mL) | Formation |
| Antibody 1 | 50 | No |
| Antibody 2 | 90 | No |
| Antibody 3 | 100 | No |
| Antibody 4 | 150 | No |
| Antibody 5 | 125 | Yes |
| Antibody 6 | 150 | Yes |
| Antibody 7 | 175 | Yes |
| Antibody 8 | 200 | Yes |
| Antibody 9 | 200 | Yes |
| Antibody 10 | 200 | Yes |
| Antibody 11 | 250 | Yes |

Organic solvents such as ethanol (EtOH) and acetone are widely used to precipitate proteins. However, organic solvents can lead to gel formation. To determine if organic solvents could be used to precipitate the antibodies prior to chromatography, the antibody-containing formulations were incubated with EtOH. As shown in Table 2 below, many of the antibodies failed to precipitate and gel formation was observed (Table 2). The limited success of using organic solvent such as ethanol to precipitate proteins restricts the application of this protein precipitation method as a universal strategy.

TABLE 2
PS80 recovery in antibody-containing formulations
after EtOH precipitation.

| Antibody-containing | PS80 recovery (%) | | Gel |
|---|---|---|---|
| formulation | (EtOH precipitation) | Precipitation | Formation |
| Antibody 1 | N/A | No | No |
| Antibody 2 | N/A | No | No |
| Antibody 3 | N/A | No | No |
| Antibody 4 | 109% | Yes | No |
| Antibody 5 | N/A | No | No |
| Antibody 6 | N/A | No | No |
| Antibody 7 | N/A | No | No |
| Antibody 8 | 107% | Yes | No |
| Antibody 9 | N/A | No | No |
| Antibody 10 | N/A | Yes | Yes |
| Antibody 11 | 118% | Yes | No |

N/A: PS80 was not analyzed in the samples as no precipitation formed.

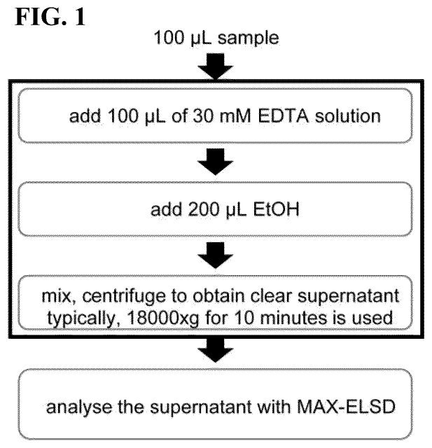

To determine if EDTA could aid in the organic solvent-induced precipitation of antibodies, the antibody-containing formulations were first mixed in 1:1 volume ratio with 30 mM EDTA and followed by mixing in 1:1 volume ratio with EtOH. Sample volumes can be, for example, 100 μL of sample, 100 μL of 30 mM EDTA and 200 μL of EtOH. The volumes of the sample, the chelating agent, and the alcohol can be scaled up proportionally. Precipitation occurred without extended incubations. An exemplary EDTA/EtOH precipitation and removal procedure is shown in FIG. 1. The combination of EDTA and EtOH resulted in the efficient precipitation and acceptable polysorbate recoveries (having an analytical target of 80-120%) for each tested antibody-containing formulation (Table 3).

TABLE 3
PS80 recovery in 11 antibody-containing formulations
after EDTA/EtOH precipitation

| Antibody- | | | |
|---|---|---|---|
| containing | PS80 recovery (%) | | Gel |
| formulation | (EDTA/EtOH precipitation) | Precipitation | formation |
| Antibody 1 | 104% | Yes | No |
| Antibody 2 | 86% | Yes | No |
| Antibody 3 | 102% | Yes | No |
| Antibody 4 | 111% | Yes | No |
| Antibody 5 | 107% | Yes | No |
| Antibody 6 | 106% | Yes | No |
| Antibody 7 | 109% | Yes | No |
| Antibody 8 | 109% | Yes | No |
| Antibody 9 | 110% | Yes | No |
| Antibody 10 | 105% | Yes | No |
| Antibody 11 | 111% | Yes | No |

% recovery greater than 100% due to the margin of error.

Although only three antibody-containing formulations precipitated upon addition of EtOH alone (Table 2), the addition of EDTA to the samples prior to EtOH precipitation (Table 3) resulted in protein precipitation for all antibody-containing formulations that were tested (Table 3). Thus, EDTA improved the EtOH precipitation process. EDTA/EtOH precipitation was a highly effective strategy to analyze polysorbate in samples with high protein concentration that could not otherwise be analyzed with the existing MAX-ELSD methods due to viscosity, gel formation, column clogging, or peak interference.

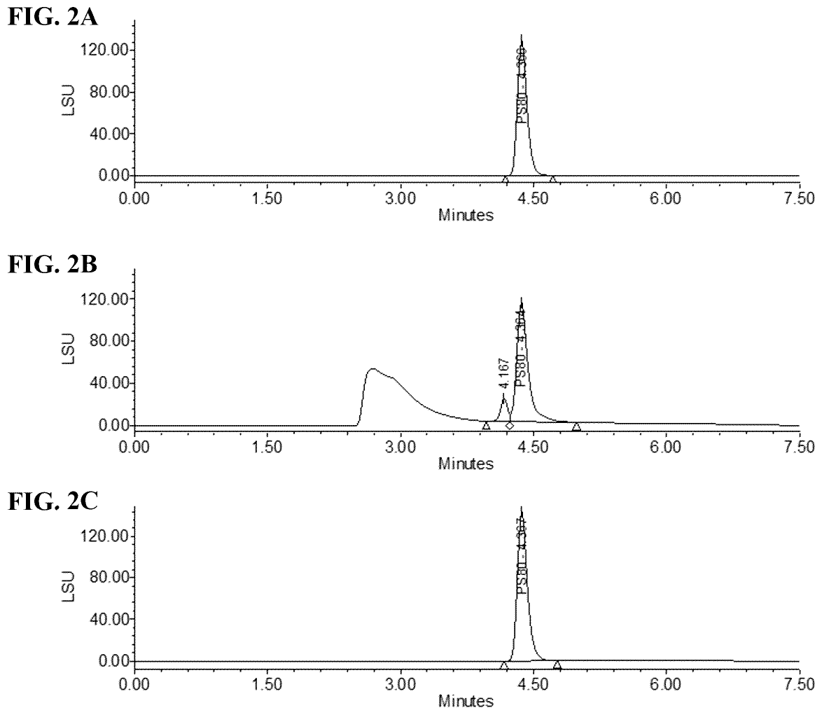

MAX-ELSD was used to analyze polysorbate-containing Antibody 4 samples. FIG. 2A shows a MAX-ELSD chromatogram of a PS80 standard, which shows a single peak. The analysis of polysorbate-containing Antibody 4 samples showed the presence of peaks eluting between 2.5 to 7.5 minutes, with one peak eluting at 4.2 minutes and potentially interfering with the PS80 peak (FIG. 2B). Removing the protein from the sample by EDTA/EtOH precipitation prior to PS80 analysis resulted in no interfering peak and improved PS80 recovery (FIG. 2C). Precipitation of protein via the EDTA/EtOH method prior to MAX-ELSD resulted in a chromatogram that resembled the PS80 standard (FIG. 2A).

The efficient protein removal by EDTA/EtOH precipitation was further demonstrated by measuring the protein concentration in the supernatant after precipitation by SoloVPE®, a variable pathlength spectrophotometer. Protein concentration in all antibody-containing formulations tested showed at least a 100-fold reduction in the amount of antibody in the solution (Table 4). As a consequence, the performance of the PS80 method was improved when analyzing samples with high protein concentration (FIG. 2C). Good recovery of PS80 using the EDTA/EtOH precipitation method was shown by the recovery within a desired range of 80%-120% of spiked PS80 relative to the antibody-containing formulations (Table 3).

TABLE 4
Antibody concentration in the supernatant after EDTA/EtOH
precipitation measured with SoloVPE ®

| | Ab concentration (mg/mL) | |
|---|---|---|
| Antibody-containing | Prior to EDTA/EtOH | After EDTA/EtOH |
| formulation | precipitation* | precipitation |
| Antibody 1 | 50 | 0.4 |
| Antibody 2 | 90 | 0.6 |
| Antibody 3 | 100 | 0.6 |
| Antibody 4 | 150 | 0.8 |
| Antibody 5 | 125 | 1.2 |
| Antibody 6 | 150 | 0.5 |
| Antibody 7 | 175 | 0.8 |
| Antibody 8 | 200 | 0.7 |
| Antibody 9 | 200 | 0.6 |
| Antibody 10 | 200 | 0.5 |
| Antibody 11 | 250 | 0.6 |

*Target concentration of polysorbate in the drug product

The performance of the EDTA/EtOH precipitation method was next evaluated by performing a test method validation for PS8 in Antibody 7-containing formulations according to the ICH guideline (www.ema.europa.eu/enich-q2r2-validation-analytical-procedures) (Table 5). This test method has also been successfully validated for Antibody 8 PS20 and Antibody 5 PS2n analysis.

TABLE 5
Performance of Antibody 7 PS80 method with EDTA/EtOH precipitation procedure

| Validation | | | | |
|---|---|---|---|---|
| Parameter | Acceptance Criteria | Results | | Pass/Fail |
| Specificity | No Interfering Peaks >1 | No Interfering Peaks >1 mV at the | | Pass |
| | mV at the retention | retention time of PS80 peak | | |
| | time of PS80 peak in the | | | |
| | worst-case matrix | | | |
| Precision - | % RSD (n = 6): ≤10% | Analyst 01 Day 01 | | Pass |
| Repeatability | | % RSD = 1% | | |
| | | Analyst 01 Day 02 | | |
| | | % RSD = 1% | | |
| | | Analyst 02 Day 01 | | |
| | | % RSD = 5% | | |
| | | Analyst 02 Day 02 | | |
| | | % RSD = 1% | | |
| Precision - | % RSD (n = 24): ≤15% | 4% | | Pass |
| Intermediate | | | | |
| Precision | | | | |
| | | Level | Average Recovery | |
| | | (μg/mL) | (%) | |
| Accuracy | Average recovery for | 75 | 95 | Pass |
| | each level is 80-120% | 150 | 104 | |
| | | 400 | 103 | |
| | | 500 | 103 | |
| | | 600 | 102 | |
| Sample Linearity | r² of measured | r² of measured concentration/ | | Pass |
| | concentration/theoretical | theoretical concentration is 1.00 | | |
| | concentration ≥0.99 | | | |
| Range | Repeatability, accuracy, | Meets for the range of | | Pass |
| | and linearity criteria are | 75 μg/mL-600 μg/mL PS80 | | |
| | met at all levels | | | |

TABLE 6
Performance of Antibody 8 PS20 method
with EDTA/EtOH precipitation procedure

| Validation | | | Pass/ |
|---|---|---|---|
| parameter | Acceptance Criteria | Results | Fail |
| Specificity | No Interfering Peaks >1 mV at | No Interfering Peaks >1 mV | Pass |
| | the retention time of PS20 peak | at the retention time of PS20 | |
| | in the PS20-free DS/DP. | peak | |
| Precision - | RSD (n = 6): ≤10% | 1% | Pass |
| Repeatability | | | |
| Precision - | RSD (n = 24): ≤15% | 3% | Pass |
| Intermediate | | | |
| Precision | | | |
| Accuracy | Average recovery for each level | 95-99% | Pass |
| | is 80-120% | | |
| Sample | r² of measured concentration/ | r² of measured concentration/ | Pass |
| Linearity | theoretical concentration ≥0.99 | theoretical concentration is | |
| | | 1.00 | |
| Range | Consecutive levels in the range | Meets for the range of | Pass |
| | should meet the criteria for | 120 μg/mL-600 μg/mL | |
| | Accuracy, Precision and | PS20 | |
| | Linearity. | | |

TABLE 7
Performance of Antibody 5 PS20 method with EDTA/EtOH precipitation procedure

| Validation | | | Pass/ |
|---|---|---|---|
| parameter | Acceptance Criteria | Results | Fail |
| Specificity | No interfering peaks >1 mV at the | No interfering peaks >1 mV at | Pass |
| | retention time of PS20 peak in the | the retention time of PS20 peak | |
| | PS20-free DS. | | |
| Precision - | RSD (n = 6): ≤10% | 1% | Pass |
| Repeatability | | | |
| Precision - | RSD (n = 24) ≤15% | 3% | Pass |
| Intermediate | | | |
| precision | | | |
| Accuracy | Average recovery for each level is | 104-112% | Pass |
| | 80-120% | | |
| Sample | r² ≥ 0.99 | r² of measured concentration/ | Pass |
| Linearity | | theoretical concentration is 1.00 | |
| Range | Consecutive levels in the range | Meets for the range of | Pass |
| | should meet the criteria for | 120 μg/mL-600 μg/mL PS20 | |
| | Accuracy, Precision and Linearity | | |

Additional chelating agents were tested for the ability to assist in the precipitation of an Antibody 7 from the antibody-containing formulation. As shown below, each of triethylamine (TEA), (ethylene glycol-bis(β-aminoethyl ether)-N,N,N′,N′-tetraacetic acid) (EGTA), n-(2-hydroxyethyl)ethylenediamine-N,N′,N (HEDTA), nitrilotriacetic acid (NTA), and 2-hydroxybenzoic acid (salicylic acid; SA) precipitated the antibody as effectively as EDTA.

TABLE 8
Analysis of additional chelating agents

| | | % Recovery of |
|---|---|---|
| | | polysorbate 80 |
| | Polysorbate 80 | relative to EDTA |
| | concentration | precipitated |
| Chelator | (μg/mL) | sample |
| Triethylamine (TEA) | 341 | 100% |
| (ethylene glycol-bis(β- | 353 | 104% |
| aminoethyl ether)-N,N,N′,N′- | | |
| tetraacetic acid) (EGTA) | | |
| N-(2- | 357 | 105% |
| hydroxyethyl)ethylenediamine- | | |
| N,N′,N (HEDTA) | | |
| nitrilotriacetic acid (NTA) | 357 | 105% |
| 2-hydroxybenzoic acid (Salicylic | 350 | 103% |
| acid; SA) | | |
| Ethylenediaminetetraacetic acid | 340 | 100% |
| (EDTA) | | |
| NaOH (Control) | no precipitation | NA |
| Formic acid (control) | gelation | NA |
| water (control) | no precipitation | NA |

The presence of high protein concentrations in biopharmaceutical products can result in chromatographic peak interference, affecting the accuracy of polysorbate analysis by MAX-ELSD. A universal and easy-to-execute protein precipitation method was developed herein. EDTA-mediated EtOH precipitation (EDTA/EtOH precipitation) removed monoclonal antibody (mAb) protein from the sample prior to HPLC-ELSD analysis, allowing successful and accurate polysorbate quantification in biopharmaceuticals with high protein concentration (≥100 mg/mL).

The introduction of the EDTA/EtOH precipitation step prior to MAX-ELSD resulted in an improved and accurate determination of polysorbate content in a wider range of samples. Protein removal by EDTA/EtOH precipitation, in comparison to precipitation with organic solvents alone, had a wider application due to the presence of EDTA, which aids protein removal by promoting precipitation and preventing gel formation. Due to its simplicity and robustness, this sample preparation method was also suitable to be applied in a quality control environment and also offered improved test method specificity and accuracy. EDTA/EtOH precipitation was simple, fast, and easy to execute. It was completed in three simple steps within a short period of time. This method was suitable for a quality control environment where polysorbate concentration will be monitored by testing the formulation prior to its release to the market and during its shelf-life, ensuring product safety and efficacy.

Those skilled in the art will appreciate that numerous changes and modifications can be made to the preferred embodiments disclosed herein and that such changes and modifications can be made without departing from the spirit of the invention. It is, therefore, intended that the appended claims cover all such equivalent variations as fall within the true spirit and scope of the invention.

What is claimed:

1. A method comprising, adding to a composition that comprises a protein and a polysorbate, an amount of a chelating agent and an amount of a C1-C6 alcohol that together are effective to precipitate said protein.

2. A method comprising:
providing a composition that comprises a protein and a polysorbate; and
adding to said composition an amount of a chelating agent and an amount of C1-C6 alcohol that is effective to precipitate said protein.

3. The method of claim 1 or 2, wherein the chelating agent is added to the composition before the alcohol is added to the composition.

4. The method of any one of the preceding claims, wherein said chelating agent is one or more of ethylenediaminetetraacetic acid (EDTA), triethylamine (TEA), (ethylene glycol-bis(β-aminoethyl ether)-N,N,N′,N′-tetraacetic acid) (EGTA), n-(2-hydroxyethyl)ethylenediamine-N,N′,N (HEDTA), nitrilotriacetic acid (NTA), and 2-hydroxybenzoic acid (salicylic acid; SA).

5. The method of any one of the preceding claims, wherein the concentration of chelating agent is from about 5 mM to about 50 mM.

6. The method of any one of the preceding claims, wherein said C1-C6 alcohol is ethanol.

7. The method of any one of the preceding claims, wherein the concentration of alcohol is from about 45% to about 55% by volume.

8. The method of any one of the preceding claims, wherein the protein is an antibody.

9. The method of claim 8, wherein the antibody is a monoclonal antibody.

10. The method of any one of the preceding claims, further comprising determining a concentration or an amount of said polysorbate in a non-precipitated portion of the composition.

11. The method of claim 10, wherein the concentration or amount of polysorbate is determined via mixed-mode anion exchange-hydrophobic high-performance liquid chromatography using an evaporative light scattering detector (MAX-ELSD).

12. The method of claim 10 or 11, wherein the method comprises, prior to determining a concentration or an amount of said polysorbate, separating the precipitated protein from the non-precipitated portion thereby forming a supernatant.

13. The method of claim 12, wherein the chelating agent, alcohol, and the composition comprising protein and polysorbate are mixed prior to separating the precipitated protein from the non-precipitated portion.

14. The method of claim 12 or 13, wherein separating the precipitated protein from the non-precipitated portion comprises centrifugation.

15. The method of claim 14, wherein the centrifugation is performed until the supernatant is essentially free of the precipitated protein.

16. The method of any one of the preceding claims, wherein the C1-C6 alcohol is ethanol and the chelating agent is selected from EDTA, TEA, EGTA, HEDTA, NTA, and salicylic acid.

17. The method of any of the preceding claims, wherein the C1-C6 alcohol is ethanol and the chelating agent is EDTA.
